(19) United States
(12) Patent Application Publication
(10) Pub. No.: US 2018/0355409 A1
(43) Pub. Date: Dec. 13, 2018

(54) TUMOR MUTATION BURDEN BY QUANTIFICATION OF MUTATIONS IN NUCLEIC ACID

(71) Applicant: GENETICS RESEARCH, LLC, D/B/A ZS GENETICS, INC., Wakefield, MA (US)

(72) Inventor: Anthony P. Shuber, Wakefield, MA (US)

(21) Appl. No.: 16/007,531

(22) Filed: Jun. 13, 2018

Related U.S. Application Data
(60) Provisional application No. 62/672,269, filed on May 16, 2018.
(60) Provisional application No. 62/526,091, filed on Jun. 28, 2017.
(60) Provisional application No. 62/519,051, filed on Jun. 13, 2017.

(51) Int. Cl. C12Q 1/6806; C12N 9/22; C12N 15/11; C12Q 1/6848

(52) U.S. Cl. CPC C12Q 1/6806; C12N 9/22; C12N 2310/20; C12Q 1/6848; C12N 2800/80; C12N 15/11

(57) ABSTRACT

The invention provides methods of developing a tumor mutation burden by detecting and quantifying target analytes in a nucleic acid sample. The sample is subjected to enrichment, which may include negative enrichment and positive enrichment. Negative enrichment may include digestion of nucleic acids that do not contain the segments. Positive enrichment may include purification of the segments. Detection and analysis of the segments may include identifying one or more mutations, sequencing, and quantifying the mutations. Relationships between the mutations may be determined and may include determining a ratio between each mutation and the plurality of mutations to develop a tumor mutation burden. The method may include a report from a clinician, where the clinician uses the tumor mutation burden for medical diagnostic purposes.

### CROSS-REFERENCE TO RELATED APPLICATIONS

This application claims the benefit of, and priority to, U.S. Application No. 62/672,269 filed on May 16, 2018, U.S. Application No. 62/526,091 filed on Jun. 28, 2017, and U.S. Application No. 62/519,051 filed on Jun. 13, 2017, the contents of each of which are incorporated by reference.

### TECHNICAL FIELD

The disclosure generally relates to molecular genetics.

### BACKGROUND

An important issue in molecular diagnostics is ensuring that a target analyte is present in a sample when the sample undergoes testing and analysis. This is difficult when the target occurs infrequently or in low abundance. For example, genetic mutations may be rare and may occur in low abundance, usually less than 1%. If information is desired for a particular mutation, but that mutation is not present in a sufficient amount in a sample, the target may not be detected.

Testing and analysis methods used today have difficulty detecting low-abundance mutations. For instance, next-generation sequencing (NGS) platforms may require sequence variants to be present at concentrations that are greater than 1% in order to be detected. In addition, due to the stochastic nature of PCR, it is possible that the mutation of interest may not be present in the sample after amplification. Therefore, ensuring the content of the tested sample, and that an area of interest is contained in that sample, is imperative when analysis results are desired for that area of interest.

### SUMMARY

The ratios of one low-abundance mutation to other mutations found in a gene may be determined to develop a tumor mutation burden for a patient. The detection and study of low-abundance or rarely occurring mutations and the relationship between the mutations have an impact in the medical diagnostics field. Clinicians may study the results from detection and analysis of certain mutations and provide prognoses for patients based on those results. As non-limiting examples, the results may allow clinicians to predict efficacy of a particular course of treatment, determine the stage of cancer, risk of metastasis, risk of reoccurrence, or monitor progression of cancer or another disease. As such, determining the presence and quantity of infrequently occurring mutations or mutations in low abundance may have significant impacts in the field of medical diagnostics.

When using analysis methods such as NGS and PCR, the area of interest, such as a mutation, may not be present in the sample. Therefore, such analysis using those methods would not be relevant to the target mutation or mutations. Further, if the analysis is not relevant to the mutation, the relationship or ratio of mutations within the sample may not be determined and the tumor mutation burden may not be determined. Knowledge of the tumor mutation burden, or the mutational landscape of a tumor, may be used to inform treatment decisions, monitor therapy, detect remissions, or combinations thereof. For example, the tumor mutation burden may be predictive of success of immunotherapy in treating a tumor, and thus methods described herein may be used for treating a tumor. As such, a report by a clinician may include a description of a plurality of mutations and an estimate of a tumor mutation burden for a tumor.

The methods of this invention include ensuring that the mutation is present in the sample, as well as providing quantification of the mutation or mutations. In an embodiment, the invention provides methods for detecting and quantifying at least one mutation in a nucleic acid sample. The sample may be obtained from a patient. The methods include protecting a segment that includes a mutation by binding a protein to the mutation and another protein to the segment, digesting unprotected nucleic acid, detecting the segment, sequencing the segment, and quantifying the segment. The methods also include optionally enriching the segment after digesting unprotected nucleic acid. The target nucleic acid may comprise a plurality of mutations. Each mutation may be detected and quantified. In the quantification step, the relationship, or ratio, between each mutation and the plurality of mutations may be determined in order to develop a tumor mutation burden for a patient. Such a ratio may have significant impacts for medical diagnostics. As such, the methods may include a clinician or healthcare professional providing a report and analysis from the quantification results to a patient. For example, the report may specify a recommended course of treatment based on the quantification results and development of the tumor mutation burden.

Embodiments of the invention use proteins to bind to the target in a sequence-specific manner. Proteins that are originally encoded by genes that are associated with clustered regularly interspaced short palindromic repeats (CRISPR) in bacterial genomes may be used. Preferred embodiments use a CRISPR-associated (Cas) endonuclease. For such embodiments, the binding protein in a Cas endonuclease is complexed with a guide RNA (gRNA) that targets the Cas endonuclease to a specific sequence. The complexes bind to the specific sequences in the nucleic acid segment by targeting a portion of the guide RNAs. When the Cas endonuclease/guide RNA complex binds to a nucleic acid segment, the complex protects that segment from digestion. Digestion may occur by one or more exonucleases. When two Cas endonuclease/guide RNA complexes bind to a segment, they protect both ends of the segment, and exonuclease can be used to promiscuously digest un-protected nucleic acid, leaving behind the segment of DNA between two bound complexes.

Embodiments of this invention use enrichment to confirm the mutations of interest are in the sample. Preferably, the enrichment is negative enrichment or negative-positive enrichment. Where a target nucleic acid comprises a first mutation and a second mutation, each mutation may be protected by a Cas/guide RNA complex. Unprotected nucleic acids are then digested, e.g. by using an exonuclease, leaving the at least one protected nucleic acid bound to the protein. This process is referred to as negative enrichment.

In negative-positive enrichment, positive enrichment follows the negative enrichment. Any suitable method may be used for the positive enrichment. The positive enrichment may include separating the protected segment from some or all of the unprotected nucleic acid. The positive enrichment may include binding the protected segment to a particle. The particle may include magnetic or paramagnetic material. The positive enrichment may include applying a magnetic field to the sample. The particle may include an agent that binds to a protein bound to an end of the segment. The agent may be an antibody or fragment thereof. The positive enrichment may include chromatography. The positive enrichment may include applying the sample to a column. The positive enrichment may include separating the protected segment from some or all of the unprotected nucleic acid by size exclusion, ion exchange, or adsorption. The positive enrichment may include gel electrophoresis.

After digestion, the protected segment of nucleic acid may be detected or analyzed by any suitable method. Detecting the nucleic acid may include identifying a mutation in the nucleic acid. Identifying the mutation may include sequencing the nucleic acid (e.g., on an NGS instrument), allele-specific amplification, and hybridization. Preferably, the target nucleic acid is amplified. Detecting the at least one target nucleic acid may further include hybridizing the target nucleic acid to a probe or to a primer for a detection amplification step, or labelling the target nucleic acid with a detectable label. The nucleic acid may be detected or analyzed by hybridization, spectrophotometry, sequencing, electrophoresis, amplification, fluorescence detection, chromatography, DNA staining, fluorescence resonance energy transfer, optical microscopy, electron microscopy, others, or combinations thereof.

Aspects of the invention provide a method for detecting a mutation. The method includes protecting a segment of a nucleic acid in a sample by introducing first Cas endonuclease/guide RNA complex that binds to a mutation in the nucleic acid and a second such complex that also binds to the same nucleic acid. The first and second Cas endonuclease/guide RNA complexes bind to the nucleic acid to define and protect a segment of the nucleic acid. Due to the mutation-specific binding of at least the first complex, the Cas/gRNA complexes only bind to, and protect, the segment in the presence of the mutation. The method includes digesting unprotected nucleic acid and detecting the segment, thereby confirming the presence of the mutation. The digesting step may include exposing the unprotected nucleic acid to one or more exonucleases.

The target nucleic acid may be quantified. The invention allows for the relationships of the mutations within the sample to be determined. For example, mutations within the sample may be compared, and a ratio between mutations within the sample may be determined. In particular, a benefit of using Cas as the binding protein is the availability of empirical data from consistent binding of the Cas protein. From the empirical data due to the consistent binding of Cas, it is possible to determine how much of the mutation is in the sample. For example, the binding efficiency of a particular Cas/guide RNA complex programmed to bind to mutation A is known. This allows for determination of how much of mutation A is in the sample, or quantification of mutation A.

As a simplified example, a Cas/guide RNA complex programmed to bind to mutation A may have a binding efficiency of 50%. After enrichment, the bound amount of mutation A may be 10 mols. Factoring in the known binding efficiency of 50%, the amount of mutation A in the sample may be calculated as 20 mols. A second Cas/guide RNA complex may be programmed to bind to mutation B and have a binding efficiency of 80%. After enrichment, the bound amount of mutation B may be 10 mols. Factoring in the known binding efficiency of 80%, the amount of mutation B in the sample may be calculated as 12.5 mols.

It is also possible to determine a relationship of the mutations in the sample. For example, presence of mutations in the sample may be compared and a ratio between two mutations may be determined. In the above simplified example, the ratio of mutation A to mutation B is 1.6 to 1. This relationship or ratio may have a significant diagnostic impact. For example, such a ratio of mutation A to mutation B may indicate a higher risk of metastasis or a higher risk of reoccurrence. Such a ratio may also indicate that a particular course of treatment may be more effective. A clinician may use results of the methods herein to identify a treatment based on the presence of the first mutation or presence of the second mutation. A clinician may also use results of the methods herein to identify a treatment based on the ratio between the mutations. Therefore, methods of the invention may include providing a report to a patient.

The nucleic acid may be any naturally-occurring or artificial nucleic acid. The nucleic acid may be DNA, RNA, hybrid DNA/RNA, peptide nucleic acid (PNA), morpholine and locked nucleic acid (LNA), glycol nucleic acid (GNA), threose nucleic acid (TNA), or Xeno nucleic acid. The RNA may be a subpopulation of RNA, such as mRNA, tRNA, rRNA, miRNA, or siRNA. Preferably the nucleic acid is DNA.

The feature of interest may be any feature of a nucleic acid. Preferably, the feature may be a mutation. For example and without limitation, the feature may be an insertion, deletion, substitution, inversion, amplification, duplication, translocation, or polymorphism. The feature may be a nucleic acid from an infectious agent or pathogen. For example, the nucleic acid sample may be obtained from an organism, and the feature may contain a sequence foreign to the genome of that organism.

The segment may be from a sub-population of nucleic acid within the nucleic acid sample. For example, the segment may contain cell-free DNA, such as cell-free fetal DNA or circulating tumor DNA.

The target nucleic acid may include a mutation specific to a tumor. The tumor mutation is present at no more than about 0.01% among matched normal, non-tumor nucleic acid.

The nucleic acid sample may be from any source of nucleic acid. The sample may be a liquid or body fluid from a subject, such as urine, blood, plasma, serum, sweat, saliva, semen, feces, or phlegm. The sample may be a liquid biopsy. The sample may comprise maternal plasma, and the nucleic acid may further comprise fetal DNA.

Each protein may independently be any protein that binds a nucleic acid in a sequence-specific manner. Preferably, the protein may be a programmable nuclease. For example, the protein may be a CRISPR-associated (Cas) endonuclease, zinc-finger nuclease (ZFN), transcription activator-like effector nuclease (TALEN), or RNA-guided engineered nuclease (RGEN). The protein may be a transcription activator-like effector (TALE). The protein may be complexed with a nucleic acid that guides the protein to an end of the segment. For example, the protein may be a Cas endonuclease in a complex with one or more guide RNAs. Preferably, catalytically inactive Cas, or d-Cas, is used. d-Cas will not exhibit nuclease activity, but will act to bind and protect the target, or mutation, from the exonuclease digestion.

The unprotected nucleic acid may be digested by any suitable means. Preferably, the unprotected nucleic acid is digested by one or more exonucleases.

### BRIEF DESCRIPTION OF THE DRAWINGS

FIG. 1 diagrams a method of quantifying a target mutation according to an embodiment of the invention including negative enrichment.

FIG. 2 diagrams a method of quantifying a target mutation according to an embodiment of the invention including negative-positive enrichment.

FIG. 3 illustrates operation of the negative enrichment method.

FIG. 4 illustrates a kit according to the present invention.

### DETAILED DESCRIPTION

The invention provides methods of detecting and quantifying nucleic acids within a sample to develop a tumor mutation burden. By performing enrichment steps, specifically negative enrichment or negative-positive enrichment, the methods allow detection and analysis of nucleic acids present at low abundance in a sample. Detection of the nucleic acids may include identifying one or more mutations. The mutations may then be analyzed and quantified and relationships between the mutations may be determined. A clinician may use such relationships for medical diagnostic purposes.

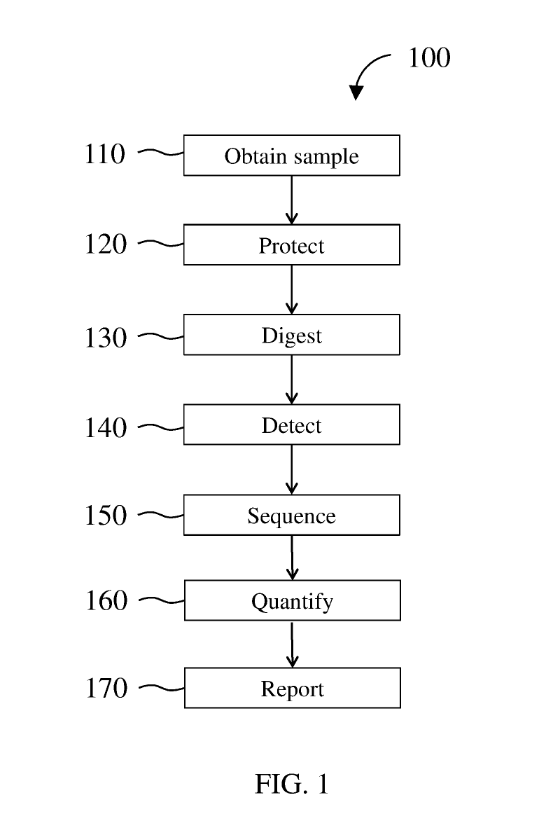

FIG. 1 diagrams a method 100 of detecting and quantifying a nucleic acid where negative enrichment is included. The method 100 may include obtaining 110 a nucleic acid sample. The method 100 further includes protecting 120 a segment in the sample by binding proteins to ends of the segment. The method 100 includes a negative enrichment step of digesting 130 unprotected nucleic acid. The method 100 then includes detecting 140 the protected segment. The method 100 includes sequencing 150 the segment. The method 100 then includes quantifying 160 the protected segment. The method may include reporting 170 the quantification results of the segment present in the sample.

For this example, two mutations of interest (mutation A and mutation B) are in the nucleic acid. As such, a first Cas/guide RNA complex is programmed to bind to mutation A and may have a binding efficiency of 40%. After negative enrichment, the bound amount of mutation A detected is 10 mols. Factoring in the known binding efficiency of 40%, it is possible to calculate that the amount of mutation A in the sample is 25 mols. Therefore, mutation A has been quantified.

Further, a second Cas/guide RNA complex is programmed to bind to mutation B and may have a binding efficiency of 80%. After negative enrichment, the bound amount of mutation B detected is 10 mols. Factoring in the known binding efficiency of 80%, it is possible to calculate that the amount of mutation B in the sample is 12.5 mols. Therefore, mutation B has been quantified.

During the quantification step, it is also possible to determine a relationship of the mutations in the sample. In the above simplified example, the ratio of mutation A to mutation B is 2 to 1. This relationship or ratio may have a significant diagnostic impact. For example, such a ratio of mutation A to mutation B may indicate a higher risk of metastasis. Such a ratio may also indicate that a particular course of treatment may be more effective. Therefore, the quantification of mutation A and mutation B and any subsequent relationship determined between the mutations may have a significant impact for diagnostic purposes. This significant diagnostic impact may then be reported, such as in a report to a patient from a clinician reviewing the quantification and using it for diagnostic purposes.

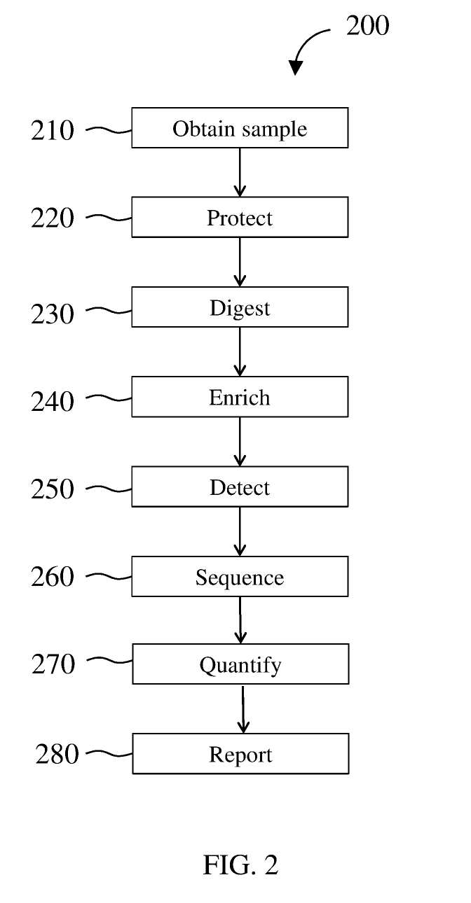

FIG. 2 diagrams a method 200 of detecting and quantifying a nucleic acid where two enrichments are conducted, namely a negative enrichment and a positive enrichment. Performing two enrichments may allow for detection of nucleic acids present at low abundance in a sample. The method 200 may include obtaining 210 a nucleic acid sample. The method 200 further includes protecting 220 a segment in the sample by binding proteins to ends of the segment. The method 200 includes a negative enrichment step of digesting 230 unprotected nucleic acid. The method 200 includes a positive enrichment step of enriching 240 the sample for the protected segment. The method 200 then includes detecting 250 the protected segment. The method 200 then includes sequencing 260 the segment. The method 200 includes quantifying 270 the protected segment. The method may include reporting 280 the quantification results of the segment present in the sample.

For this example, a particular Cas/gRNA complex programmed to bind to mutation A may have a binding efficiency of 40%. After negative enrichment, the bound amount of mutation A is 10 mols. Factoring in the known binding efficiency of 40%, it is possible to calculate that the amount of mutation A in the sample is 25 mols.

Further, a second Cas/gRNA complex may be programmed to bind to mutation B and have a binding efficiency of 50%. After negative enrichment, the bound amount of mutation B is 10 mols. Factoring in the known binding efficiency of 50%, it is possible to calculate that the amount of mutation B in the sample is 20 mols.

It is also possible to determine a relationship of the mutations in the sample. In the above simplified example, the ratio of mutation A to mutation B is 1.25 to 1. This relationship or ratio may have a significant diagnostic impact. For example, such a ratio of mutation A to mutation B may indicate a higher risk of metastasis or a higher risk of reoccurrence. Such a ratio may also indicate that a particular course of treatment may be more effective.

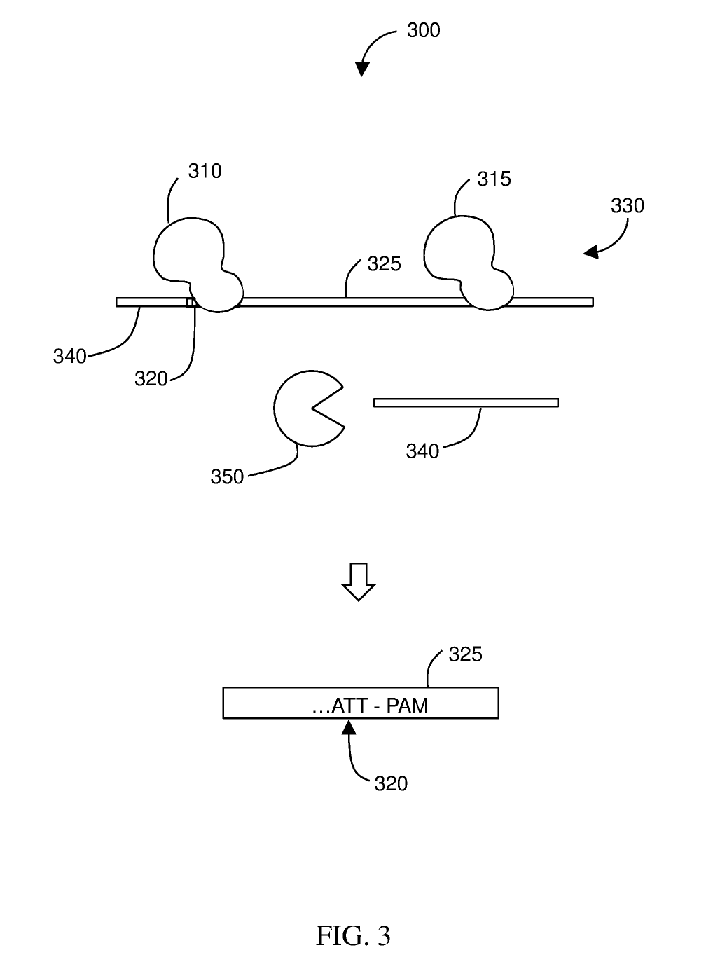

FIG. 3 illustrates operation of negative enrichment. The sample 300 includes DNA 305 from a subject. The sample 300 is exposed to a first Cas endonuclease/guide RNA complex 310 that binds to a mutant fragment 325 mutation in a sequence-specific fashion. Specifically, the complex 315 binds to the mutation 320 in a sequence-specific manner. A segment of the nucleic acid 330, i.e., the mutant fragment 325, is protected by introducing the first Cas endonuclease/guide RNA complex 310 (that binds to a mutation in the nucleic acid) and a second Cas endonuclease/guide RNA complex 315 that also binds to the nucleic acid. Unprotected nucleic acid 340 is digested. For example, one or more exonucleases 350 may be introduced that promiscuously digest unbound, unprotected nucleic acid 340. While the exonucleases 350 act, the segment containing the mutation of interest, the mutant fragment 325, is protected by the bound complexes 310, 315 and survives the digestion step intact.

The described steps leave a reaction product that includes principally only the mutant segment 707 of nucleic acid, as well as any spent reagents, Cas endonuclease complexes, exonuclease 350, nucleotide monophosphates, and pyrophosphate as may be present. Optionally, a positive enrichment may be carried out following the negative enrichment. The positive enrichment allows the segment to be separated from other nucleic acids that are not removed by the digestion step. For example, some nucleic acids may not be fully degraded during the digestion, so they may interfere with detection of the segment. Any suitable method of purification or enrichment may be used.

The methods include detecting the segment 330 (which includes the mutation 320). Any suitable technique may be used to detect the segment 330. For example, detection may be performed using DNA staining, spectrophotometry, sequencing, fluorescent probe hybridization, fluorescence resonance energy transfer, optical microscopy, electron microscopy, others, or combinations thereof. Detecting the mutant segment 325 indicates the presence of the mutation in the subject (i.e., a patient). For example, hybridization, spectrophotometry, sequencing, electrophoresis, amplification, fluorescence detection, chromatography, DNA staining, fluorescence resonance energy transfer, optical microscopy, electron microscopy, others, or combinations thereof may be used for detection of the mutant segment.

The method may further include providing a report describing the mutation in the patient. The report may include describing the presence of the mutation or mutations. The report may also include describing the quantity of the mutation or mutations. The report may include a description of the relationship or ratio between one mutation and another mutation. The report may include a course of treatment recommended by a clinician based upon, for example, review of the presence of the mutation and relationship or ratio of one mutation to another mutation.

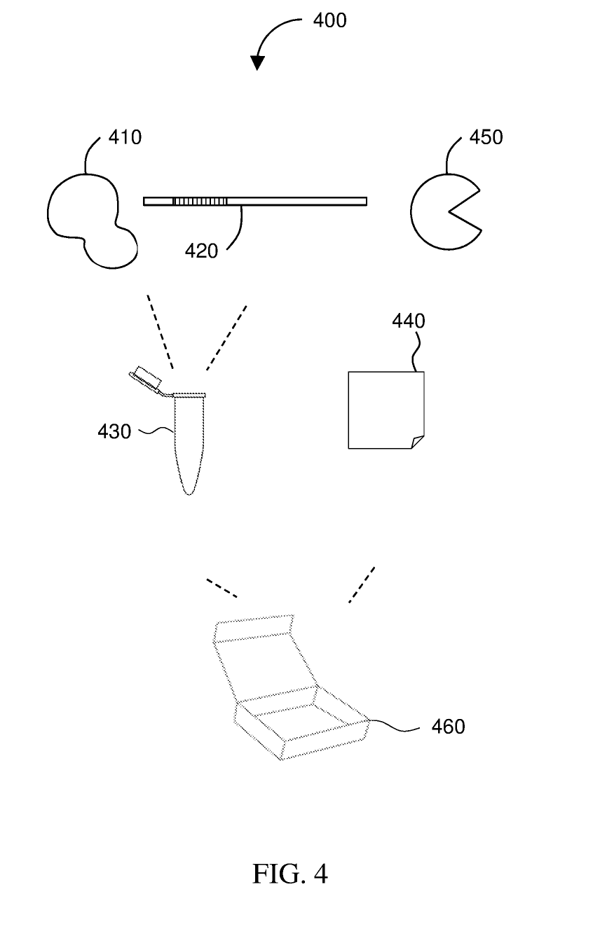

FIG. 4 shows a kit 400 of the invention for carrying out the methods of this invention. The kit 400 may include reagents 903 for performing the steps described herein. For example, the reagents 430 may include one or more of a Cas endonuclease 410, a guide RNA 420, and exonuclease 450. The kit 400 may also include instructions 440 or other materials, such as pre-formatted report shells that receive information from the methods to provide a report (e.g., by uploading from a computer in a clinical services lab to a server to be accessed by a geneticist in a clinic to use in patient counseling). The reagents 430, instructions 440, and any other useful materials may be packaged in a suitable container 460.

Kits of the invention may be made to order. For example, an investigator may use, e.g., an online tool to design guide RNA and reagents for the performance of the methods herein. The guide RNAs 420 may be synthesized using a suitable synthesis instrument. The synthesis instrument may be used to synthesize oligonucleotides such as gRNAs or single-guide RNAs (sgRNAs). Any suitable instrument or chemistry may be used to synthesize a gRNA.

In some embodiments, the synthesis instrument is the MerMade 4 DNA/RNA synthesizer from Bioautomation (Irving, Tex.). Such an instrument can synthesize up to 12 different oligonucleotides simultaneously using 50, 200, or 1,000 nanomole prepacked columns. The synthesis instrument can prepare a large number of guide RNAs 420 per run. These molecules (e.g., oligos) can be made using individual prepacked columns (e.g., arrayed in groups of 96) or well-plates. The resultant reagents 430 (e.g., guide RNAs 420, endonuclease(s) 410, exonucleases 450) can be packaged in a container 460 for shipping as a kit.

In certain aspects, the disclosure provides a method for determining and reporting a tumor mutational burden for a tumor. The method includes obtaining a sample comprising tumor DNA, wherein the tumor DNA comprises a plurality of mutations. The method includes isolating fragments of the tumor DNA via DNA isolation methods with empirically known or demonstrable success rates. For example, a negative enrichment may be performed by using a Cas endonuclease or catalytically inactive homolog thereof (“Cas proteins”). Each Cas protein can be provided with a guide RNA that binds to, or near, a specific tumor mutation. Pairs of the Cas proteins each bind to ends of a segment of the tumor DNA that contains a mutation. While the pairs of Cas proteins are bound to the segments and protecting the segment, other unbound DNA is digested promiscuously in the sample using exonuclease. After the Cas proteins are incubated with the sample comprising the tumor DNA and the negative enrichment via exonuclease is performed, the tumor DNA that was protected by Cas protein is assayed (e.g., detected or sequenced) to determine an identity and frequency for each of a plurality of mutations. For each identified mutation, its count—or frequency—is extrapolated using a reciprocal of a binding rate for the associated Cas protein and the corrected mutations counts are summed across the Cas proteins/targets to predict a mutational burden level for the tumor.

The binding rate of each Cas protein is known or determined empirically (e.g., by testing in vitro on synthetic DNA or amplicons in controlled conditions using qPCR to quantify what percentage of Cas protein successfully binds to its cognate target). Exemplary binding rates may include showing, for example, that Cas protein A (in a complex with a guide RNA) binds to 60% of available target (leaving 40% of valid cognate targets unbound); Cas protein B binds to 15% of target that is available; Cas protein C binds to 50% of available target; while Cas D binds to 95% of available target. Without being bound by any mechanism or theory, it may be that different binding rates are a product of guide RNA design and guide RNA designs are constrained by the requirement to minimize false positives and correct for false negatives using the empirically-determined binding efficiency. Thus in the foregoing example, Cas protein D may bind with high (95%) efficiency due to, e.g., an entire 20 base target stretch adjacent the PAM being wholly unique within the genome and also being GC rich. In contrast, the hypothesized Cas protein B may bind to only 15% of available target if, say, the target includes a repeating genome motif that is also found frequently outside of the intended target. Using methods of the disclosure, the off-target binding is of minimal concern as the binding efficiencies provide ratios for corrected what is measured to have bound in the sample.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method of developing a tumor mutation burden based on quantification of mutations in a target nucleic acid, the method comprising:
obtaining a sample comprising a target nucleic acid, the target nucleic acid comprising a plurality of mutations;
detecting the plurality of mutations in the sample to obtain a detected plurality of mutations;
sequencing the detected plurality of mutations from the sample to obtain a sequenced plurality of mutations;
quantifying the sequenced plurality of mutations to obtain a quantified plurality of mutations; and
developing a tumor mutation burden for the sample based on ratios between mutations in the plurality of mutations determined by using the quantified plurality of mutations.

2. The method of claim 1, wherein detecting the plurality of mutations further comprises:
binding a plurality of proteins to the plurality of mutations of the target nucleic acid in a sequence-specific manner;
digesting non-target nucleic acid in the sample; and
dissociating the plurality of bound proteins, leaving the detected plurality of mutations.

3. The method of claim 2, wherein quantifying further comprises:
determining an amount of each mutation in the target nucleic acid based on a binding efficiency of a protein to the mutations.

4. The method of claim 3, wherein developing a tumor mutation burden further comprises:
determining a ratio between each mutation and the plurality of mutations by using the amounts of each mutation.

5. The method of claim 1, further comprising amplifying the plurality of mutations.

6. The method of claim 5, wherein amplifying is carried out by qPCR.

7. The method of claim 1, wherein the sample is from a patient, and the method further comprises providing a report describing the tumor mutation burden as present in the patient.

8. The method of claim 7, further comprising identifying a treatment based on the tumor mutation burden in the patient and including the identified treatment option in the report.

9. The method of claim 2, wherein the digesting step comprises exposing the non-target nucleic acid to one or more exonucleases.

10. The method of claim 2, wherein the plurality of proteins each comprise an RNA-guided protein complexed with a guide RNA, the guide RNA comprising a targeting portion that hybridizes to a complementary portion in the target nucleic acid.

11. The method of claim 10, wherein the RNA-guided protein comprises a Cas endonuclease.

12. The method of claim 11, wherein the Cas endonuclease is catalytically inactive.

13. The method of claim 1, further comprising performing an enrichment to obtain a representative portion of the target nucleic acid from the sample.

14. The method of claim 13, wherein the enrichment is a negative enrichment.

15. The method of claim 13, wherein the enrichment is a negative-positive enrichment.

16. The method of claim 1, wherein the sample is a blood sample, serum sample, plasma sample, urine sample, saliva sample, semen sample, feces sample, phlegm sample, or liquid biopsy.

17. The method of claim 1, wherein the target nucleic acid includes a mutation specific to a tumor.

18. The method of claim 1, wherein the sample comprises maternal plasma, and wherein nucleic acid comprises fetal DNA.

19. The method of claim 2, wherein the detecting step further comprises one selected from the group consisting of hybridization, spectrophotometry, sequencing, electrophoresis, amplification, fluorescence detection, and chromatography.

20. The method of claim 1, wherein detecting the target nucleic acid includes hybridizing the target nucleic acid to a probe or to a primer for a detection amplification step, or labelling the target nucleic acid with a detectable label.
